(19) United States
(12) Patent Application Publication
(10) Pub. No.: US 2026/0229146 A1
(43) Pub. Date: Aug. 6, 2026

(54) SYSTEMS, PLATFORMS, AND METHODS OF INJECTION TRAINING

(71) Applicant: Truinject Corp., Newport Beach, CA (US)

(72) Inventors: Gabrielle A. Rios, Fullerton, CA (US); Scott Cameron Royston, Austin, TX (US); Clark B. Foster, Mission Viejo, CA (US); Matthew Ryan, Aliso Viejo, CA (US); James Neesen, Irvine, CA (US)

(21) Appl. No.: 19/640,316

(22) Filed: Apr. 6, 2026

Related U.S. Application Data
(63) Continuation of application No. 16/118,324, filed on Aug. 30, 2018, now abandoned.
(60) Provisional application No. 62/552,307, filed on Aug. 30, 2017.
(60) Provisional application No. 62/553,729, filed on Sep. 1, 2017.
(60) Provisional application No. 62/643,388, filed on Mar. 15, 2018.

(51) Int. Cl. G09B 23/28; A61M 5/46; A61M 5/48; G09B 5/00; G09B 5/02; G09B 23/30

(52) U.S. Cl. CPC G09B 23/285; A61M 5/46; A61M 5/48; G09B 5/00; G09B 5/02; G09B 23/30

(57) ABSTRACT

An injection system or platform can simulate outcome of injection procedures, which can be specific to the patients'ethnicities, to aid the injector and patients in selecting a treatment plan. An injection system or platform can also include a pair of augmented reality glasses that allow a user of the system to visualize underlying anatomy, a needle tip underneath a patient's skin, and/or injection sites on the patient. An injection service provider and patient matching system can facilitate better matching between the injection service providers and the patients, which can improve the injection outcome for the patients.

### INCORPORATION BY REFERENCE TO ANY PRIORITY APPLICATIONS

This application is a continuation of U.S. patent application Ser. No. 16/118,324, filed Aug. 30, 2018, which claims benefit of U.S. Provisional Application No. 62/552,307, filed Aug. 30, 2017, U.S. Provisional Application No. 62/553,729, filed Sep. 1, 2017, and U.S. Provisional Application No. 62/643,388, filed Mar. 15, 2018, the entirety of each of which is hereby incorporated by reference.

Any and all applications for which a foreign or domestic priority claim is identified in the Application Data Sheet as filed with the present application are hereby incorporated by reference under 37 CFR 1.57.

### FIELD OF THE DISCLOSURE

The present disclosure generally relates to improving injection outcomes and augmented reality injection aid tools and injection systems or platforms for providing aid to a physician or healthcare professional during injection procedures for training and for live patients.

### BACKGROUND

A variety of medical injection procedures are often performed in prophylactic, curative, therapeutic, or cosmetic treatments. Injections may be administered in various locations on the body, such as under the conjunctiva, into arteries, bone marrow, the spine, the sternum, the pleural space of the chest region, the peritoneal cavity, joint spaces, and internal organs. Injections can also be helpful in administering medication directly into anatomic locations that are generating pain. These injections may be administered intravenously (through the vein), intramuscularly (into the muscle), intradermally (beneath the skin), subcutaneously (into the fatty layer of skin), or by way of intraperitoneal injections (into the body cavity). Injections can be performed on humans as well as animals. The methods of administering injections typically vary for different procedures and may depend on the substance being injected, the needle size, or the area of injection.

Injections are not limited to treating medical conditions, such as cancer and dental treatment, but may be expanded to treating aesthetic imperfections, restorative cosmetic procedures, procedures for treating migraine, depression, lung aspirations, epidurals, orthopedic procedures, self-administered injections, in vitro procedures, or other therapeutic procedures. Many of these procedures are performed through injections of various products into different parts of the body. The aesthetic and therapeutic injection industry includes two main categories of injectable products: neuromodulators and dermal fillers. The neuromodulator industry commonly utilizes nerve-inhibiting products such as Botox®, Dysport®, and Xeomin®, among others. The dermal filler industry utilizes products administered by providers to patients for orthopedic, cosmetic and therapeutic applications, such as, for example, Juvederm®, Restylane®, Belotero®, Sculptra®, Artefill®, Voluma®, Kybella®, Durolane®, and others. The providers or injectors may include plastic surgeons, facial plastic surgeons, oculoplastic surgeons, dermatologists, orthopedist, primary care givers, psychologist/psychiatrist, nurse practitioners, dentists, and nurses, among others.

### SUMMARY

The current state of the art utilizes a syringe with a cylindrical body and a plunger that moves within the body. The body has a discharge end that can attach to a needle or IV for delivery of medication. The healthcare professional or any one performing an injection on a patient (“injector”) relies on his or her experience to locate an injection site on the patient, such as on the face of the patient, and/or to perform a particular injection procedure at a certain needle angle, depth, dosage, etc. The injector is also not able to view the needle tip and/or the underlying anatomy after the needle has punctured the patient's skin. Therefore, the effect of the injection can vary based on the injector's experience, preferred techniques, and/or the like.

In addition, it is hard to visualize the effect of an injection on the patient. Patients can have difficulty in selecting a treatment plan without being able to visualize beforehand the effect of an injection.

Moreover, patients'anatomy, including but not limited to the skin and the underlying anatomy, can vary among the population. Factors relating to a person's appearance can include age, ethnicity, genetic composition, life style, and the like. These factors not only can affect the short-term outcome of an injection, but also can affect the long-term effect of the injection, such as due to the rate the patient's body metabolize the medication or injected material. In the context of cosmetic or aesthetic injections, patients can also have varying expectations of the procedure, which can be related to the patients'perception of beauty, for example, due to ethnicity and/or cultural influences, and/or to the patients'treatment goal, for example, whether a patient wants to remedy aesthetic imperfections, and/or to look younger and more refreshed. It can also be beneficial to have a training tool for injectors to train on ethnicity-specific injection techniques, such as different injection sites on patients of different ethnicities for the same type of injection.

The present disclosure provides various systems, platforms, and methods of injection training and for providing injection to live patients. The injection training platform can include an injection syringe and a training apparatus. The injection syringe can have a plunger configured to move relative to a syringe barrel containing an injection material or compound. The syringe can have a needle configured to deliver the injection material or compound into an injection location on the training apparatus.

The plunger, syringe barrel, and/or needle can have one or more sensors, such as position and/or orientation sensors, force and/or pressure sensors, time of flight sensors, and/or other types of sensors. The training apparatus can have an appearance of a patient's anatomy, such as a head, torso, limbs, and the like. The training apparatus can have a plurality of cameras inside a cavity of the training apparatus. The plurality of cameras can be spaced apart from one another. The cameras can detect a light source from the needle tip. A hardware processor is configured to determine a position, such as a three-dimensional position, of the needle tip based on the detected light source. The processor can be on the syringes, the training apparatus, and/or a remote server.

The system can have a training module. The training module can receive data about the injection procedure performed by a trainee using the injection syringe on the training apparatus. The system can record data about the injection procedure performed by a trainee using the injection syringe on a live patient. The training module can provide training instructions, scoring, feedback, and/or evaluation of an injection procedure, and/or certification of the trainee, for example, when the trainee has passed a test for an injection technique.

The embodiments described below are compatible with and can be part of the embodiments described in U.S. Published Application No. 2017/0254636, U.S. Published Application No. 2017/0245943, U.S. Published Application No. 2017/0136185, and/or U.S. Published Application No. 2018/0211562, the entirety of each of which is incorporated herein by reference. Some or all of the features described below can be used or otherwise combined together or with any of the features described in U.S. Published Application No. 2017/0254636, U.S. Published Application No. 2017/0245943, and/or U.S. Published Application No. 2018/0211562.

The present disclosure also provides an injection service provider and patient matching system can facilitate better matching between the injection service providers and the patients. The patient can search for an injection service provider based on one or more criteria, such as proximity, qualification, type of procedures performed, or others. The patient can book appointments with the injection service providers and/or make payments using the matching system. The patient can rate the injection service provider on a plurality of factors, such as professionalism, level of satisfaction with the outcome, or otherwise. The injection service provider can also rate the patient. The matching system can encourage patients to return to the same service provider. The same service provider can perform more consistent injection procedures on the patient, which can improve the injection outcome. The patient can also pay a premium for “VIP” services. The patient can take photos so that the system can archive information about the injections, photos so that the system can store information such as how much products have been injected, the injection locations. The injection provider can use the information to try to replicate the same injection at the next visit. Sharing of post-injection photos can also aid in mitigating risks of complications.

The present disclosure provides an injection training platform comprising a computing system having at least one processor and a memory device. The computing system can be configured to receive and process inputs relating to locations of an injection tool relative to a training anatomical model so as to output at least a virtual training anatomical model. The computing system can be configured to apply one or more visual filters to the virtual training anatomical model so that the computing system further outputs to an augmented reality viewing system at least one of training technique instructions or an underlying anatomy overlaying the virtual anatomical model. The one or more filters can comprise ethnic-based anatomical variations. The platform can comprise a display unit in electrical communication with the computing system, wherein the display unit displays a depiction of the training injection technique.

In some embodiments of the platform, the inputs can be from an injection tool and a training anatomical model, the injection tool and/or the training anatomical model in electrical communication with the computing system. In some embodiments of the platform, the inputs can be from a joystick, the joystick in electrical communication with the computing system.

In some embodiments of the platform, the filter can further comprise a genetic makeup filter or a Phi ratio filter.

The present disclosure provide a method of providing injection training using an injection training platform having an injection tool and an anatomical model. The method can comprise collecting scans of internal anatomy of patients; processing the scans so as to form a database of ethnicity-specific patient anatomies; and using the database to provide ethnicity-specific injection training.

In some embodiments, the method can further comprise outputting virtual images of ethnicity-specific patient anatomies based on the database to an augmented reality viewing system. The virtual images can be projected over the anatomical model.

In some embodiments, the method can further comprise outputting to the augmented reality viewing system ethnicity-specific injection training instructions.

In some embodiments of the method, the scans can comprise MRI scans, CT scans, photos, and/or X-ray images.

The present disclosure provides an injection platform configured to recommend a treatment plan to a live patient. The platform can comprise a computing system having at least one processor and a memory device. The computing system can be configured to receive and process inputs relating to a patient's anatomy so as to output a virtual patient model. The computing system can be configured to receive and process information relating to the patient's past and present appearances so that the computing system further recommends and outputs a simulated outcome of an injection procedure on the virtual patient model. The simulated outcome can be configured to show a more natural look of the patient.

In some embodiments of the platform, the computing system can be further configured to apply one or more filters to the virtual patient model.

In some embodiments of the platform, the one or more filters can comprise an ethnicity filter, a patient preference filter, a genetic makeup filter, and/or an activity level filter.

In some embodiments of the platform, the computing system can recommend injection procedures based on Phi ratios.

In some embodiments of the platform, the simulated outcome can comprise outcomes of recommend and alternative injection techniques and/or products.

In some embodiments of the platform, the simulated outcome can be used for training on an anatomical model.

In some embodiments of the platform, the simulated outcome can comprise short term and long term effects of an injection procedure and/or product.

The present disclosure provides an injection platform comprising a computing system having at least one processor and a memory device. The computing system can be configured to generate an augmented environment for display on a pair of augmented reality glasses in electrical communication with the computing system. The pair of augmented reality glasses can be configured to allow a user of the system to view an injection target with the augmented environment. The platform can comprise an augmented reality projection configured to provide at least a virtual visual representation of a portion of an internal anatomy of a patient's body portion overlaid or projected onto a patient's body portion and synchronized with movements of the patient's body portion.

In some embodiments of the platform, the computing system can communicate with a smart watch worn by a user wearing the pair of augmented reality glasses and performing an injection procedure. The smart watch can convey injection information by haptic feedback to the user.

In some embodiments of the platform, the augmented reality projection can further comprise simulated or actual volume of injection.

In some embodiments of the platform, the augmented reality projection can further comprise simulated effect of injection on local tissue of the patient's body portion.

In some embodiments of the platform, the simulated effect of injection on the local tissue can comprise effect of a needle penetrating an artery and/or a nerve.

In some embodiments of the platform, the computing system can be further configured to develop a recommended injection treatment plan. The recommended injection treatment plan can be based at least in part on predict outcomes of an injection procedure.

In some embodiments of the platform, the augmented reality projection can be a three-dimensional augmented reality projection showing depth.

In some embodiments of the platform, the platform can further comprise a syringe configured to measure a volume of medication delivered.

The present disclosure provides a system for matching injection providers and patients. The system can comprise a database of injection providers. The injection providers can receive training using an injection training platform comprising an injection tool, an anatomical model, and a training module. The system can comprise a database of patients. The system can comprise a processor in electrical communication with the database of injection providers and the database of patients. The processor can be configured to, in response to a patient searching for an injection provider, output a selection of injection providers, wherein the patient is allowed to view certification of the injection providers using the injection training platform.

In some embodiments of the system, the processor can be configured to search for injection providers based on proximity of the injection providers.

In some embodiments of the system, the processor can be configured to search for injection providers based on a type of procedure the injection providers are certified to provide.

In some embodiments of the system, the processor can be configured to receive ratings of the injection providers by the patients.

In some embodiments of the system, the processor can be configured to receive ratings of the patients by the injection providers.

More details of various features of the injection training platforms and/or methods are discussed below. Although the descriptions herein may be in the context of cosmetic facial injections, the embodiments described herein can be configured for use in any part of the patient's body and/or for any types of injections.

### BRIEF DESCRIPTION OF THE DRAWINGS

FIG. 1A illustrates an example injection system or platform.

FIG. 1B illustrates an example injection training module main menu interface.

FIG. 1C illustrate an example injection training module scoring and/or evaluation interface.

FIGS. 1D-1E illustrate an example training history interface of the injection training module.

FIG. 1F illustrates an example block diagram of a processor of the injection system or platform.

FIG. 1G illustrates a flow chart for an injection training process.

FIGS. 2A and 2B illustrate another example injection system or platform used on a live patient.

FIG. 2C illustrates an example projection of virtual anatomy on a training apparatus.

FIGS. 3A and 3B illustrate example facial recognition techniques.

FIG. 4 is an example front view from an augmented reality tool of the injection system or platform.

FIGS. 5A-5D illustrates example front views from an augmented reality tool of injection training platform when the patient is moving.

FIG. 6 illustrates an example smart watch to be used with an injection system or platform disclosed herein.

FIG. 7 illustrates schematically a joystick for use in injection training.

FIGS. 8-28 illustrate an example injection training service provider and patient matching system.

### DETAILED DESCRIPTION

Aspects of the disclosure are provided with respect to the figures and various embodiments. One of skill in the art will appreciate, however, that other embodiments and configurations of the devices and methods disclosed herein will still fall within the scope of this disclosure even if not described in the same detail as some other embodiments. Aspects of various embodiments discussed do not limit scope of the disclosure herein, which is instead defined by the claims following this description.

### Overview of Injection System or Platform

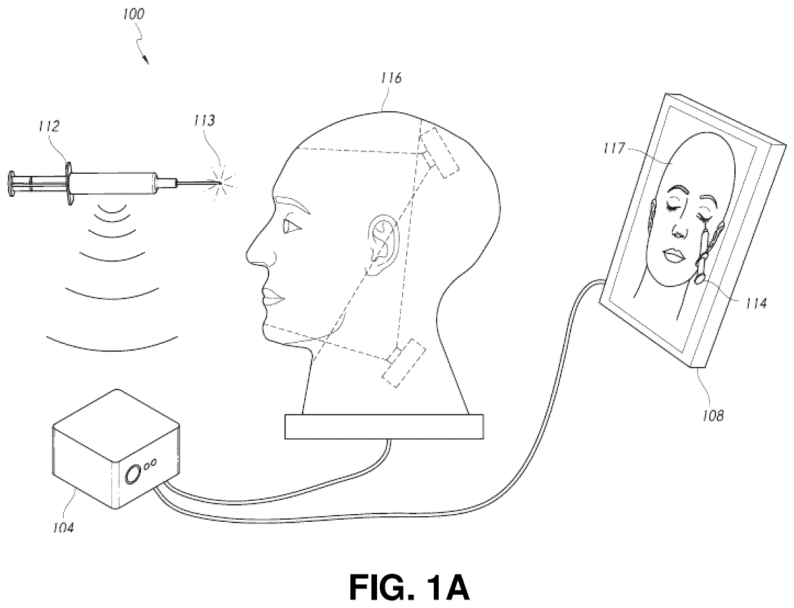

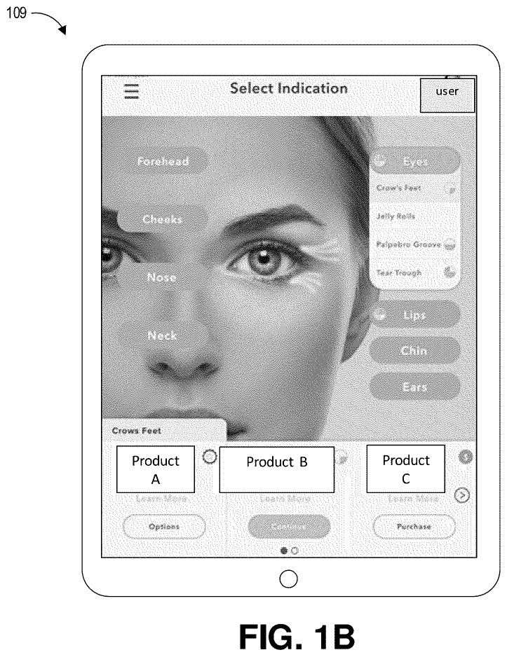

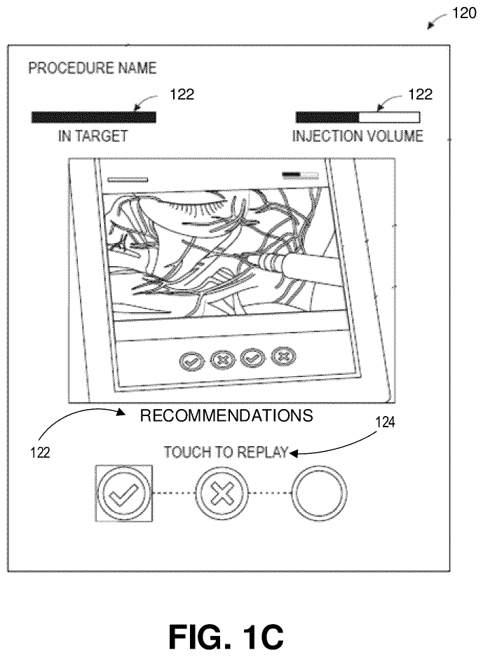

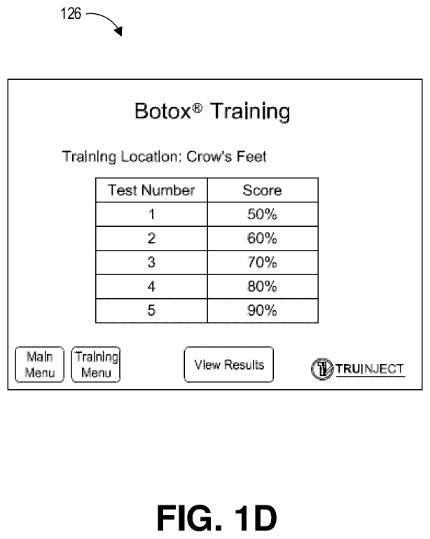

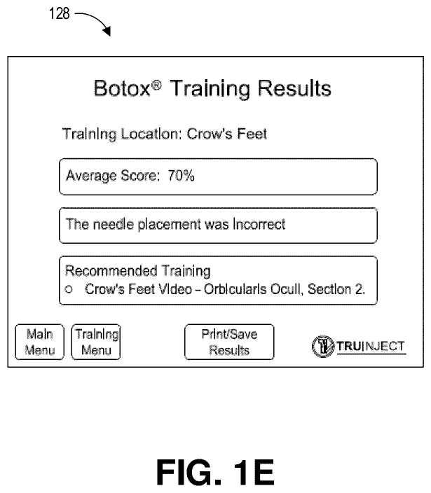

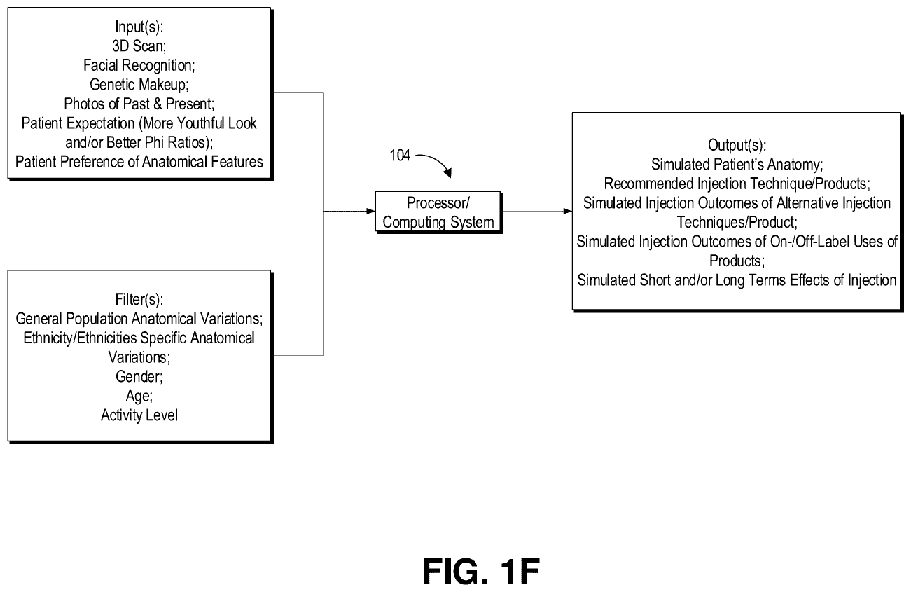

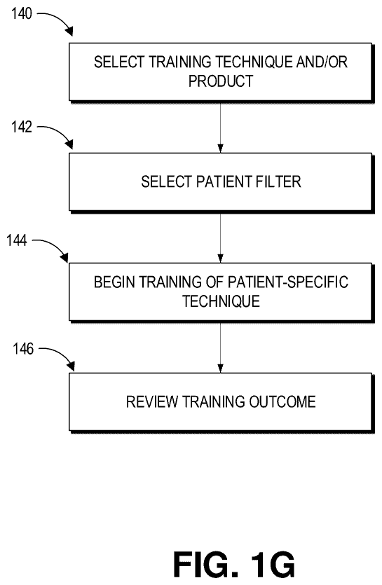

FIG. 1A illustrates an injection system or platform 100. Although the illustrated system 100 is configured for training injections, the system 100 can also be configured to perform injections on live patients, for example, by using an injection tool with location and/or position tracking tools, sensors for measuring the injection force and/or volume, and the like. Injection training platform100 can include a computing system 104 configured to generate a simulated injection procedure, a display device 108 configured to present the simulation, an injection tool 112 having a needle configured to deliver a simulated and/or actual injection by the injector. For injection training, the system 100 can also include an anatomical model 116 configured to receive the injection. The display device 108 can be coupled to the computing system 104 via a cable and/or any wireless technology. Injection training platform100 can aid the injector in more informed treatment procedures and superior outcomes.

The injection tool 112 can have one or more tracking sensors configured to measure and transmit injection information to the computing system 104. The computing system 104 can incorporate the position and orientation information into the simulated injection displayed to the injector. The injection tool 112 can include one or more sensors to measure the position and orientation in three-dimensional space of the injection tool 112. The injection tool 112 can also include one or more sensors configured to measure the amount of medication, or dosage applied during an injection. More details of example injection tools that can be included in the system 100 are described in U.S. Published Application No. 2017/0254636, U.S. Published Application No. 2017/0136185, and U.S. Published Application No. 2018/0211562, incorporated herein by reference in their entirety. The injection tool position and orientation information, dosage measurement, and/or other types of injection information can aid the computing system 104 in guiding the injector during the injection procedure.

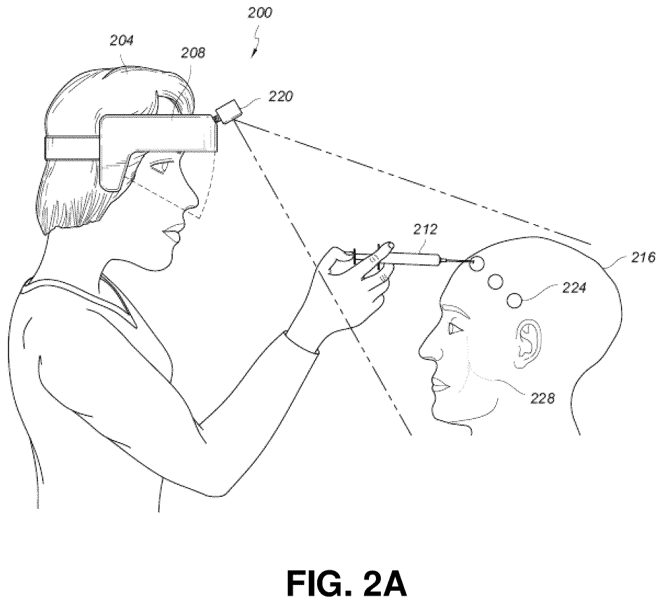

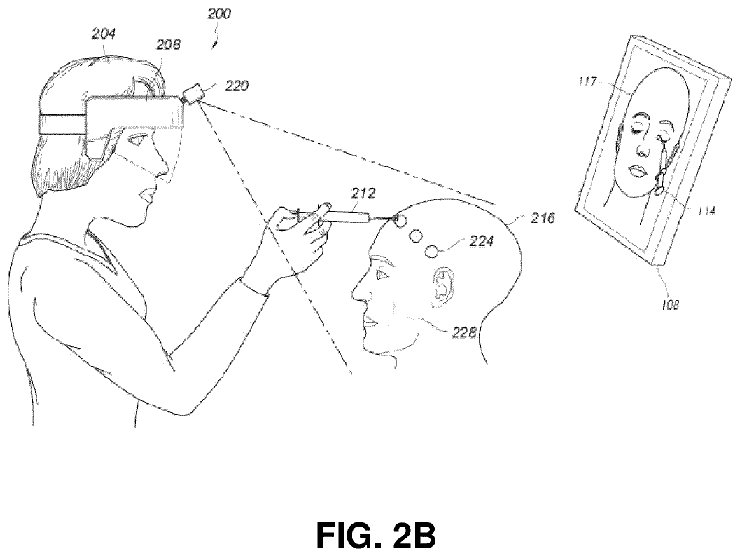

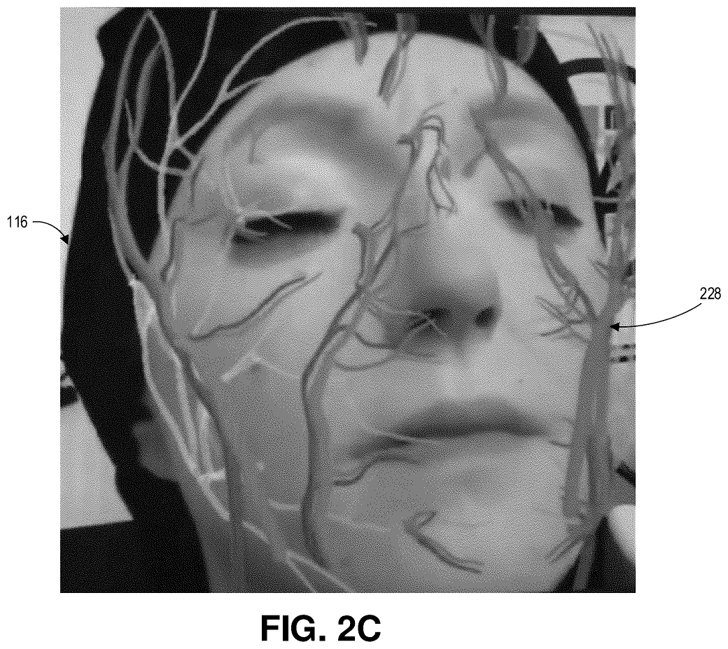

As shown in FIG. 1A, when the system is used for injection training using the anatomical model 116, the system 100 can include an optical tracking system that can aid the determination of the position and orientation of the injection tool 112 in the three-dimensional space. When injection training platform100 is used for performing injections on live patients, such as shown in FIGS. 2A-2B (described below), the injection tool may not have the optical tracking system.

The display device 108 can be on a tablet, mobile device, laptop, or standalone display. The display device 108 can also include wearable eyewear, such as spectacles, goggles, or glasses, such as shown in FIGS. 2A-2B, or as otherwise described herein. The wearable eyewear can include virtual reality (“VR”) or augmented reality (“AR”) glasses or goggles.

The display device 108 can display a simulated patient's face. The display device 108 can also display both a simulated patient's face and the anatomy on the simulated face. When the injector moves the injection tool 112, the motion of the injection tool 112 relative to the simulated patient's face can be displayed on the display device 108. Injection training platform100 can display a virtual injection tool 114 relative to the simulated patient's face 117 based on communicated positions and orientation information provided by the injection tool 112.

One or more injection sites (for example, a display of a target injection site) can be superimposed on the simulated patient's face 117 for the injector to more easily identify the desired injection location. The display device 108 can also overlay one or more computer-generated three-dimensional images on the simulated patient's face 117. The computer-generated image(s) can correspond to one or more layers of anatomy (for example, bones, nerves, blood vessels, or the like) for the specific target. The images can be obtained using a CT scan, an MRI scan, a photographic image, an X-ray, and/or the like from live patients (such as patients of the general population). The computing system 104 can overlay underlying anatomy, for example, blood vessels and nerves, over the simulated patient's face 117 to help the injector visualize areas to avoid during the injection. As the virtual tool 114 approaches and penetrates the simulated patient's face 117, various levels of the patient's underlying anatomy according to the depth of the tip of the virtual tool 114 can be displayed. The simulated injection can also include a display of the medication or therapeutic agent being delivered to the injection site. The injection tool 112 can be any of the syringes disclosed in U.S. Published Application No. 2017/0254636 and U.S. Published Application No. 2018/0211562.

### Scoring and/or Evaluation System

The training system can have a plurality of training modules. Some examples of the training modules are described in U.S. Published Application No. 2015/0262512 and U.S. Published Application No. 2017/0178540, the entirety of each of which is incorporated herein by reference. The training system can have a user interface, such as a graphic user interface (“GUI”), displaying the plurality of training modules. As shown in FIG. 1B, the plurality of training modules can be displayed on various possible target locations of a patient's anatomy in an example main menu 109. For example, the interface can display a patient's face and the user can access the plurality of training modules by touching and/or selecting a location where injection may be required, such as the nose, check, chin, forehead, or others. In some embodiments, the user can view a plurality of products available for the injection location. The layout of the main menu 109 is not limiting.

The user can touch and/or select one of the training modules to be directed to the training module interface. The training module interface can provide a plurality of options, such as viewing the anatomy, watching a training video, slides and/or other materials, taking a practice injection procedure, and/or taking an injection technique. The training video can be provided as part of the training package to which the user has access. The training video can include recorded training techniques of certain experts and can be available for purchase.

The practice training procedure can be displayed with substantially real-time scoring, feedback, and/or evaluation systems. The interface can also display a virtual tool (such as a syringe) that shows how much medication is being dispersed, whether or not the user has hit a vital structure, labels to which structures were penetrated, and/or the like. The user can view score charts and/or numbers of the location accuracy, injection force, dosage, and/or other factors as the user performs the practice injection procedure. The location accuracy scoring can be based at least in part on whether the needle has reached the target location, and/or on how close the needle tip is to a center of the target location. The user can view substantially real-time three-dimensional computer models of the training apparatus and the syringe as the injection procedure is performed. The model can include colored dots (such as the colored dots 224 in FIG. 2A) or other types of markers, such as shown in U.S. Published Application No. 2017/0178540, incorporated herein by reference in its entirety. The dots can mark the target injection locations. As the user performs the practice injection procedure, the dots can turn red or a different color when the needle hits an artery and/or is close to an artery. The dots can have different sizes indicating different desired amounts of injection material(s). In some embodiments, the dots can change in size and/or color in response to the amount of injection material(s) the user has injected into the location.

The practice injection procedure and/or the test procedure can be performed without the GUI displaying the scoring, feedback, and/or evaluation systems. The user may or may not be able to view the three-dimensional models of the training apparatus and/or the syringe during the injection procedure. FIG. 1C illustrates an example training module interface 120 during and/or after the injection procedure. After the practice injection procedure and/or the test procedure, the user can review the scoring, feedback, and/or evaluation 122 of the training procedure performed. The user can replay 124 the injection procedure and review changes in the scoring charts and/or the three-dimensional animation of the training apparatus and/or the syringe.

The injection training platform can aggregate data of multiple injection procedures from the same user and/or different users. For example, FIGS. 1D and 1E illustrate an individual user's training results for a particular procedure (such as Botox injection at the crow's feet). The training results 126 in FIG. 1D illustrates the user's scores for the particular procedure over time. The training results 128 in FIG. 1E illustrates the user's average score for the procedure, recommended area of improvement, and recommended trainings. In some embodiments, aggregated data from a plurality of doctors and/or injection experts can be configured to aid in determining an acceptable and/or standard injection location, and/or technique. In some embodiments, the recorded data of injection procedures on the training apparatus and/or live patients can be submitted to a regulatory body for an approval process of certain injection material products or syringe products.

### Accounting for Anatomical Variations Among Various Populations & Personalized Aesthetics-Based Facial Mapping

The injection training platform can also include on the display device indicators of vasculature in a patient's anatomy that can account for variations among the general population, age, sex, ethnicity, and/or the like. As shown in FIG. 1F, the processor or the computing system 104 can receive one or more of the inputs, such as user or patient inputs, inputs from a sensor or a medical equipment, or others, and/or filters, which will be described in greater detail below, to output simulations based at least in part on the one or more inputs and/or filters. The inputs can include, for example, readings from a facial recognition software, a 3D scanner (such as a CT scanner or an MRI scanner), results from a DNA sequencing chip, past and present photos of the patient, patient expectations (such as whether the patient wants to achieve a more youthful look, a more natural look, and/or improve the Phi ratios of the patient's facial features), and patient preferences of certain facial features (for example, fuller lips, etc.). The filters can include, for example, a general population filter, an ethnicity-based filter, a gender filter, an age filter, and/or a lifestyle or activity level filter. The outputs can include, for example, simulated patient's anatomy, recommendations of the injection techniques and/or products, simulation of outcomes of the injection (such as of the recommended and/or alternative techniques and/or products, on-and/or off-label uses of the products, long-term and/or short-term effects, or otherwise).

A three-dimensional computer image corresponding to the training apparatus or the patient can be displayed on the display device. The computer image can have one or more markers for an anatomical landmark, artery, vein, and/or nerve. The one or more marks can form a statistical “cloud” based on locations of the arteries, veins, and/or nerves obtained from survey(s) of patients of the general population, age, sex, ethnicity, and/or the like. The cloud can indicate a “no fly” zone, such as a three-dimensional space that the needle tip needs to avoid. In some embodiments, the one or more markers can be a plurality of (for example, 4, 5, 6, or other) discrete paths indicating typical locations of an artery, vein, and/or nerve in people of the general population, age, sex, ethnicity, and/or the like. In some embodiments, the training apparatus can include the plurality of discrete paths for different ethnic, sex, age, and/or other versions of the training apparatus. In some embodiments, the plurality of paths can aid and/or augment the scanner device described in U.S. Published Application No. 2017/0245943 in determining actual locations of an artery, veins, and/or nerves in a live patient.

The injection training platform can also include a gaming or training application, which can allow an injector, such as an injector using the injection systems or platforms described herein, to learn different injection techniques that are unique for different ethnicities. The application can optionally further allow a user, such as a doctor, a medication manufacturer, or a patient, to visualize a simulated effect of a certain injection procedure on the patient. The application can be run on a computer, a tablet, a smartphone, and/or the like. The application can be run independent of the injection systems or platforms described herein. The computing system 104 described herein can also optionally include the application.

As shown in a training process in FIG. 1G, at step 140, the user can select a training technique and/or product. At step 142, the user can select a patient filter. The filter can include any of the filters disclosed herein, such as ethnicity, patient preference, genetic makeup, and the like. As will be discussed below, the filters can also be applied when performing injections on live patients. At step 144, the user can be trained on a patient-specific technique using the training module. The training module can provide, for example, ethnicity-specific anatomical variations, demonstration of an expert technique for various patient groups, or otherwise. At step 146, the user can review the training outcome, such as by viewing side-by-side the training procedure and the expert procedure, and/or the latest training procedure and the training sessions completed in the past.

Additional details of the inputs and filters will be described below. The application can include a database of three-dimensional scans, such as MRI scans, of the anatomy of people who have received injections. The database of the application can have a catalog of the 3D scans of patients of different ethnicities, and also optionally the injection outcomes for those ethnicities. The application is described herein using facial features and facial aesthetics injections as examples, but the anatomy of interest and the relevant injection procedures in the database of the application are not limiting.

The 3D scans can be from people of various ethnicities who have received filler injections. The database can include scans of at least three to five, or at least the top 20 ethnicities, such as Caucasian, Chinese, Korean, Mexican, Eurasian, mixed ethnicity, and the like.. The application can include analysis of the differences among all the 3D scans so as to understand variations in the anatomy across different ethnicities. The application can store different injection techniques that can be applied to a particular ethnicity. For examples, the injection sites and/or the dosage of the product can differ among different ethnicities to get the best outcome based on the bone structure, facial map, muscles, artery locations, other key indicators, etc.

The database can also optionally include information on the preferences of facial features that differ among various ethnicities. The database can also optionally include information on different types of facial aesthetics outcomes, such as information about the golden ratio, or the Phi ratio calculations. For example, the database can include a model face incorporating the 23 golden ratios in the facial features.

A user of the application can use any facial recognition software to analyze a patient's face. The facial recognition software can be built into the application. The facial recognition software can run analysis of the patient's face based on one or more photos, videos, and/or live streaming of the patient.

In some implementations, the user can apply an ethnicity filter to the analysis. The filter can be selected by the application based on a determination of the patient's ethnicity by the facial recognition software, and/or by a manual selection by the user. The manual selection needs not correspond with the patient's actual ethnicity, but can be more aligned with the patient's expected outcome of the injection. For example, a user of one ethnicity may want to have an injection outcome more typical of a different ethnicity.

In some variants, an ethnicity match can also be performed by an analysis of the patient's gene sequencing. The gene sequencing can be determined by performing a DNA test using a DNA testing chip or otherwise, for example, by screening the patient's blood sample against the DNA testing chip. Other genetic information that can be useful for the application when calculating and recommending injection sites, product dosage, and/or treatment options can include the patient's metabolic rate (which can impact longevity of the medication, such as the Botox and filler), the patient's metabolism of the medication, the patient's healing attributes (such as bruising, adverse reactions, allergic reactions), and/or other medications the patient is taking that may react with the injected product.

When the patient is of a mixed ethnicity based on any of the ethnicity identification methods described herein, the application can blend the 3D scans of the respective ethnicities when applying the ethnicity filter. For example, if someone is 50% eastern European, 30% Asian, and 20% African based on DNA test results or otherwise as disclosed herein, the ethnicity filter applied by the software can be a mix of at least substantially 50% eastern European, 30% Asian, and 20% African.

When the user is an injector or a trainee of an injection procedure, the user can specify an ethnicity that the user wants to train on. The user can select the ethnicity for training based on the client base and/or the expected clients, for example, when the user wants to train and/or specialize in injections on Chinese patients. The application can display the injection sites that are unique to a certain ethnicity so that the user can learn the techniques that have an improved outcome on that ethnicity. When the application is part of injection training platform described herein, the display device can overlay the injection sites and/or underlying anatomy that are unique to that ethnicity to guide the injector in the actual injection on live patients and/or training injection procedure.

The application can also apply other types of filters, such as filters based on the patient's age, lifestyle, and the like. For example, the training system can be configured to provide personalized aesthetics-based facial mapping. The application can also ask the user to upload past and/or recent pictures of the patient. The past photos can show how the patient looked like in the previous predetermined number of years, for example, the past five or ten years. The application can instruct the user to upload at least a predetermined number of past photos, such as about five to twenty photos. The past and present photos can be helpful for analyzing how the patient's look has changed over the years. The analysis can be helpful, especially when the patient's treatment goal includes a more youthful and/or refreshed look, and/or a natural look. Information from the past and present photos can be used to determine which injection outcomes are the best match for the patient's goal.

The training system can be configured to provide simulation of an effect of an injection on the patient. The simulation can be based at least in part on data relating to how the patient looked in the past and/or how the patient look on the day of the injection and/or consultation. The simulation can show effects of various injection locations, using various techniques, and/or of various doses. The system can recommend an injection product, technique, and/or amount of the product to the patient based on the simulation. The simulation can also be based on photos taken of the patient's face at various time points. The system can provide simulation of how the patient's face can change based on the product, the technique, and/or the amount of the product. The simulation can be based at least in part on the life style of the patient, for example, the activity level, diet, sleep, or other factors, and/or hereditary factors of the patient.

After having applied the one or more filters, the application can simulate and display an injection outcome for a particular injection procedure and/or product. The application can calculate and/or recommend where and how much medication or facial aesthetics product to inject to give the patient the outcome that the patient hopes to achieve. For example, if the patient's goal is primarily or includes a more youthful look, the application can run one or more algorithms to calculate and make the recommendations based at least in part on how the patient looked in the past and how the patient looks on the day of the injection procedure and/or consultation. The application can calculate a predetermined number of data points, such as at least a million data points, on the past and recent or current photos in order to recommend treatment protocols and options. The recommendation can also be a combination of what the patient's face would look like with phi calculations and the difference between the patient's past and present looks. The application can also make recommendations based on other user inputs, such as how natural the patient wants to look so that it is not readily apparent that the patient has undergone the injection procedures. The application can recommend injection sites and/or product amounts to give the patient a range of possible outcomes. The application can also optionally predict how the patient will look a predetermined amount of time after the injection procedure, for example, based at least in part on the patient's metabolic rate, medical history, prior injection procedures, age, other genetic factors, and/or other factors.

The application can simulate both on-label and off-label simulations to simulate what the patient can look like if the patient chooses only on-label injection sites, only off-label injection sites, and/or both. The application can allow the user, such as the medication manufacturer or the doctor, to determine if the “off-label” look is preferred over the look after performing injections in the on-label injection sites.

The application can include an online community in which users, such as injectors and patients, can vote on what are the best outcomes based on crowd sourcing. The application can provide photos of the patient, with the patients'consent, before the injection and after, such as just after the injection, or photos of the simulated injection outcome. The online community can vote on the injection outcome that looks most natural, most beautiful, most youthful, most refreshed, and the like. The crowdsourcing voting can also be on the “on-label” verses “off-label” patients or any other photos of the patient post-injection to see what injection procedure gets more popular outcomes.

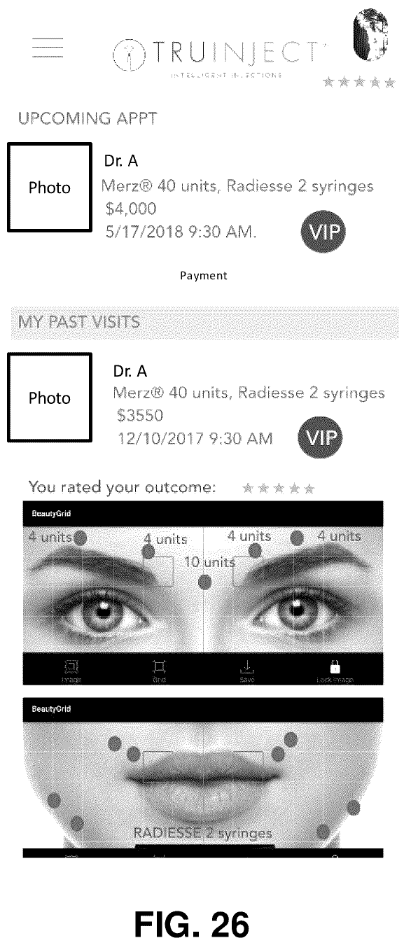

Patients who have used the application and/or received injections using the systems described herein can also set up user profiles (such as shown in FIG. 26) to track information about their procedures, satisfaction levels, how long the effect lasted, what the patients wanted to have done differently or better, and/or the like. Patients can answer online survey questions to combine their genetic data with other data points, which can further improve their personalized facial aesthetics treatment plan. The application described herein can become more accurate in simulating the outcome of injections and/or recommending treatment plans as patients add more data about the outcomes of their injections. The application can improve genetics-based calculations by better correlating the injection outcomes and genetics as more patient data is added to the database. The application can also include an online community of patients to share their injection experiences, which can promote patient education on the injection procedures, and/or improve injection safety and/or outcomes.

When the user is a trainee injector, such as performing an injection as the user sees fit on an anatomical model, (and/or when the user is performing an actual injection on the patient,) the application can simulate the outcomes for any specific ethnicity. The application can compare the outcomes based on how the injector injected (including but not limited to injection locations, angles, volume, and/or when the medication or product dispersed) and an “ideal outcome” from an expert injector. The application can recommend what the training injector should have done differently to help the injector train and improve the techniques for the specific ethnicity.

The application can display a split screen view, which can be on the display device 108 of FIG. 1, for the trainees and/or their trainers. Showing the injection outcomes of the trainee and an “expert” side by side on the split screen view can provide better contrast of the trainee's technique and the expert's technique. The application can display various viewing options showing and comparing a virtual patient's face with or without products injected, a virtual patient's after actual product injection and/or with simulated injections, and/or the like. The viewer can toggle between the options. The viewing option can include, for example, displaying side by side and/or comparing a simulated face with no products and an MRI scan of a face with no product, or a simulated face with no products and the simulated face with the injections completed, or a simulated face with no products and a simulated face with simulations of injections completed, and/or a simulated face with no products and the simulated face with recommendations of injection points, sequencing, amount of product. The technique recommendations can be provided by the application based on machine learning.

### Augmented Reality Aided Injection System or Platform

Injection training platform described herein can also include augmented reality features. FIGS. 2A and 2B illustrate an example injection system or platform 200 used on a live patient 216. Injection training platform 200 can have any of features of the injection aid system 100. Injection training platform 200 can also incorporate the application described above. Accordingly, features of injection training platform 100, features of injection training platform 200, and features of the application described above can be incorporated into one another.

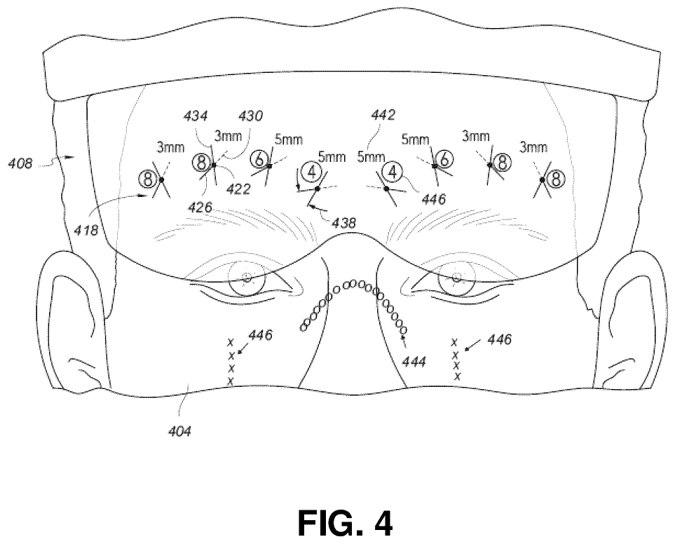

The system or platform 200 can include an injection tool 212 having a needle configured to deliver simulated and/or actual injection to a patient 216 by a user 204. The system 200 can also include an augmented reality display. By way of non-limiting examples, augmented reality displays can include wearable glasses 208, holographic projection, or other augmented reality tools permitting the user to see the physical world, while also providing computer-generated images, or an augmented environment, to be overlaid on the user's field of view. Examples of wearable glasses can include the Oculus Rift, offered by Oculus VR of Menlo Park, California, the HoloLens offered by Microsoft, or any other augmented reality glasses. The augmented reality system can be implemented using AR kits, such as the ones made by Apple or Google. The wearable glasses 208 are illustrated as having a headband with see-through glasses. FIG. 4 illustrate another example of wearable glasses 408. The form factors of the wearable glasses are not limiting. The wearable glasses can be universal or custom-made for the users. For example, the wearable glasses can have prescriptions for users whose vision is impaired due to near-sightedness, far-sightedness, astigmatism, and/or the like.

As shown in FIG. 2B, the system 200 can optionally include a separate display device 108 as shown in FIG. 1. The display device 108 can display any of the features described above with regard to the system 100, the system 200, and the application.

The wearable glasses 208 can display a virtual patient's anatomy 228, which can be derived from scans of the anatomy of the patient. The virtual patient's anatomy 228 can be projected over the actual patient 216's face. The system 200 can include a computing system configured to generate the augmented environment including, among other things, the patient's underlying anatomy. The wearable glasses 208 can be coupled to the computing system and configured to present simulated images depicting the augmented environment generated by the computing system. In some embodiments, the underlying anatomy of human patients 228 can also be projected onto a training apparatus, such as shown in FIG. 2C, which can help a trainee to improve the injection techniques. In some embodiments, the projected anatomy can be ethnicity-specific, gender specific, and/or others, so that the trainee can improve techniques that are tailored for those patient groups.

The virtual patient's anatomy can be based on a patient's specific physiology. Definition of the patient's anatomy can be obtained by, for example, a CT scan, an MRI scan, a photographic image, an X-ray, or the like, and used to form the anatomical model depicted in the augmented reality environment. For example, a patient's arteries, veins and/or nerves can be projected through augmented reality onto a patients face. In some implementations, as the patient moves their head, the projected underlying anatomy can move with facial movement.

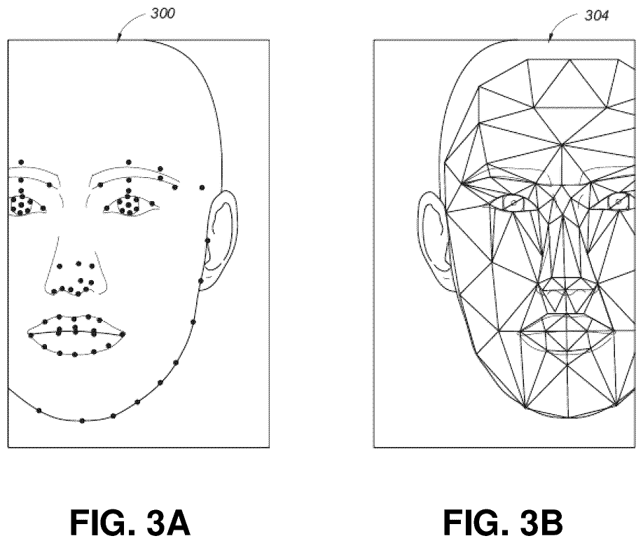

Injection training platform 200 can utilize various patient facial scans to develop a comprehensive mapping of the patient's underlying anatomy 228 to the patient's face. Images of the patient's face can be acquired through use of a camera or scanner 220 of the injection aid system 200. Facial recognition software of injection training platform 200 can spot a user based on contrast patterns typically seen in and around a human face or otherwise. FIG. 3A illustrates an example point-mask 300 generated to identify contrast patterns around a face. To obtain specificity as illustrated in FIG. 3A, injection training platform 200 can be trained using multiple faces manually marked with points to show where certain anatomical features reside. Injection training platform 200 can generate a baseline point-mask from the manually-marked faces, as illustrated in FIG. 3A. The anatomical features can include the borders of lips, eyes, nose, and face. Injection training platform 200 can incorporate the baseline point-mask to individual patients and alter the baseline point-mask 300 to generate a patient-specific point-mask 300 matching the patient's face. Injection training platform 200 can also create a mesh 304, such as shown in FIG. 3B from the point-mask 300. The mesh 304 can be capable of moving in accordance with a corresponding movement of the patient, as will be described below. The mesh 304 can also trigger a response when the patient engages in particular activity, such as blinking, smiling, opening or closing its mouth, among other actions, which will be described below. The mesh 304 can be implemented using a smartphone (such as a smartphone running on iOS or Android).

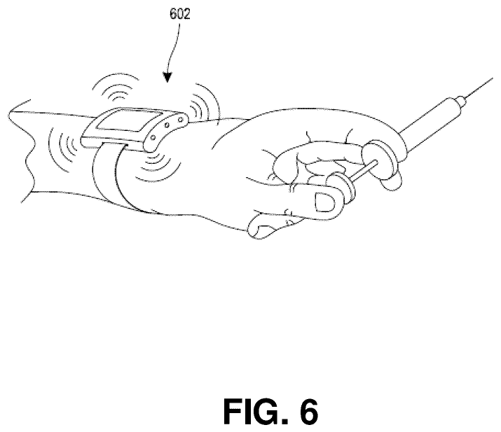

Some injection information, such as less complex information can also optionally be delivered to the injection provider using other tools or methods. For example, haptic feedback can be provided to the injector using a smart watch 602 (see FIG. 6) or similar device. Information such as volume of medication injected (a dose indicator), end of injection notification, time for a new injection site, proximity to no-go tissue (for example, nerves and/or arteries that need to be avoided), and/or effect of the injection of medication into the local tissues can be conveyed with vibration patterns detectable by an injector who is wearing the smart watch. The effect of injection of medication into the local tissues can also include occlusion of artery and/or degraded nerve functions. The haptic feedback using the smartwatch 602 can be in addition to, or alternative to, the visual indictors using the augmented reality glasses 208. A haptic feedback can be felt by the injection provider, for example, to inform the injection provider that the needle has entered a dangerous zone, without the patient knowing about it and without the patient being harmed.

The computing system can overlay the patient's underlying anatomy 228 over the captured patient's face in the injector's field of view. The computing system can also cause treatment sites 224 and/or other information configured to aid the injection to be displayed over the patient's face. The augmented reality glasses 408 can thus enable the user 404 to see the patient 216 with a projection of the patient's underlying anatomy 228 and other information superimposed on the patient's face. One or more of the patient filters disclosed above (such as the ethnicity filter, genetic makeup filter, or otherwise) can be applied to the projected anatomy 228.

As shown in FIGS. 2A and 2B, predetermined injection sites 224 for product delivery can be identified by a target 3-dimensional location, a larger allowable “sphere” around the injection site. The injection sites 224 can be unique to a specific ethnicity, as described herein. The user can also optionally see the allowable range of therapeutic product injection amounts via the glasses 208. The user can be instructed as to the region and/or set of injection sites on which to perform the procedure, as well as the amount of therapeutic product to be injected. Similarly, the system can also visually, audibly, and/or via haptic feedback (such as when used in combination with the smartwatch), warn against injecting in danger areas where the patient anatomy could cause pain or complications. For example, if an injected needle goes too deep and is close to or in an artery or near a nerve, the system can warn the user and provide instructions to pull out or not go any deeper.

As shown in FIG. 4, the wearable glasses 208 can display the information including but not limited to, preferred injection sites, with color and or shape symbols coded to indicate the medication type, volume depth of injection along with needle angle, medication delivery guides, nearby anatomy risks, or others. The anatomy risks that the injection needle should avoid can include, but are not limited to, nerves and/or arteries. The wearable augmented reality glasses 208 can project marks indicating nerves tissues 444 that the needle should avoid, and/or marks indicating artery tissues 446 that the needle should avoid. Injection training platform200 can display the under-skin anatomical features. The displayed underlying anatomy can convey critical tissue information such as arteries, veins, nerves, fat and muscles. The user can be better informed of the areas that should be avoided during the injection, such as blood vessels and nerves.

As shown in FIG. 4, the display device 408 can also display one or more treatment icons 418. Each treatment icon 418 can represent an injection site on the patient. The penetration point can be represented by a penetration indicator 422. The skin track icon 426 can represent an orthogonal projection from the surface of the patient at the injection site.

The computing system can also cause a needle tip of the injection tool 412 to be projected into the face of the patient and simulated patient's anatomy after the needle tip has punctured the patient's skin. At each time step of a simulated and/or actual injection, the amount of therapeutic product injected at the location of the needle tip can be estimated. The amount of therapeutic product injected can also be determined using, for example, a Syringe Dose and Position Device and methods of determining plunger position as described in U.S. Published Application No. 2018/0211562, incorporated herein by reference in its entirety. The underlying anatomy displayed in the user's field of view can vary based on the user's selection, such as whether the user wants to view a muscle layer, a nerves layer, a blood vessel, and/or any combinations thereof. The underlying anatomy displayed in the user's field of view can also optionally vary based on the depth of the needle. The projections can be three dimensional such that a care provider can see better the depth at which different anatomical features reside.

The wearable glasses 208 can also display the injection track as represented by the skin track icon 426. The wearable glasses 208 can also provide information relating to the angle formed between the injection tool 212 and the patient during treatment by the treatment angle icon 438. The treatment angle icon 438 can be formed between a skin track icon 426 and angle track 434. The wearable glasses 208 can also provide a depth icon 430 representing an injection depth. Injection training platform200 can represent the injection depth by a numerical depth icon 442. The wearable glasses 208 can also contain information including a volume medication to be injected, as represented by the volume indicator 442.

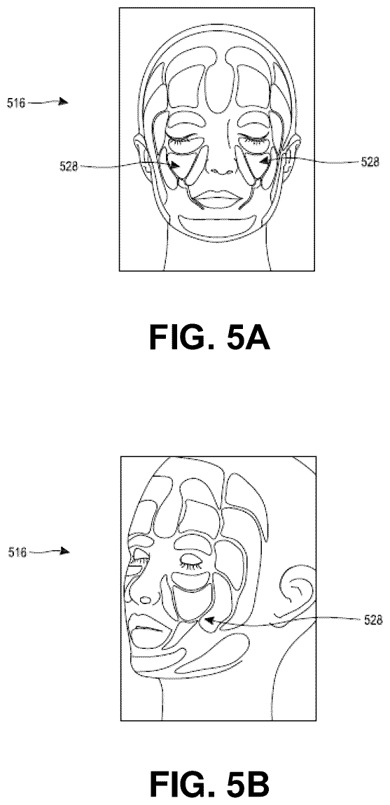

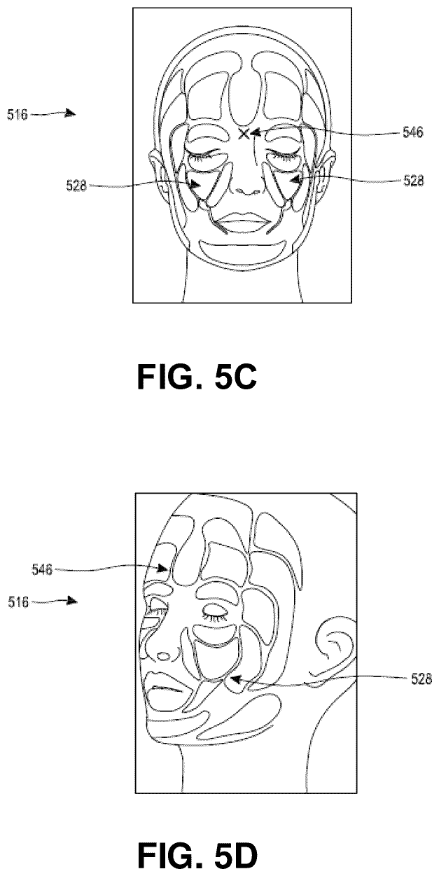

As shown in FIGS. 5A-5D, the patient's underlying anatomy 528 and/or other information disclosed herein, such as the injection sites 546, can move with the patient 516. For example, when the patient's head 516 moves sideways as shown in FIGS. 5A-5D, the underlying anatomy 528 and the injection sites 546 can move with the patient. As described above, the scanner 220 of the system or platform 200 can also track facial expressions. The patient head can be scanned multiple times while specific facial expressions are exercised so as for the computing system to understand the patient's facial expressions. The expressions can involve smiling, frowning, yawning, shrugging, laughing, resting, among others. The resulting scans can be processed to identify the underling muscles positions and actions.

The computing system can also incorporate the application described above so as to provide treatment recommendations to the patient. The treatment recommendation can be specific to the patient's ethnicity, age, life style, DNA and genetic profiles. Injection training platform200 can develop an escalating sequence of treatments from minor facial injection through major facial surgery. The recommendations can also optionally include an estimated psi ratio value for each level of treatment. The recommendations can also optionally include the volume of products needed for the patient.

An example use of injection training platform200 will be described in connection with a glabellar injection. The application described above can provide the patient with treatment recommendations, which can be unique to the patient's ethnicity, DNA data, and/or other factors. The application can provide the patient and the injector with one or more simulated injection outcomes. The patient can select any one of the treatment plan. During the injection procedure, the user, such as the injector, can wear the glasses 208. Through the glasses 208, the injector can see in his or her field of view the injection points (for example, five injection points) for the glabellar injection with the patient's underlying anatomy overlaid over the patients face. The injector can also optionally apply an ethnicity filter and/or other filters described herein to view ethnicity specific underlying anatomy and/or injection sites. The position and orientation sensor on the injection tool can allow the computing system to simulate the needle tip once the needle of the injection tool 212 has penetrated the patient's skin. The augmented reality glasses 208 can provide visualization of the patient's underlying anatomy, injection locations, areas and/or vital structures to be avoided, and/or a needle tip penetrating a portion of the underlying anatomy. The injector can perform the injection and review the procedure information, while not having to look away from the patient.

### Joystick

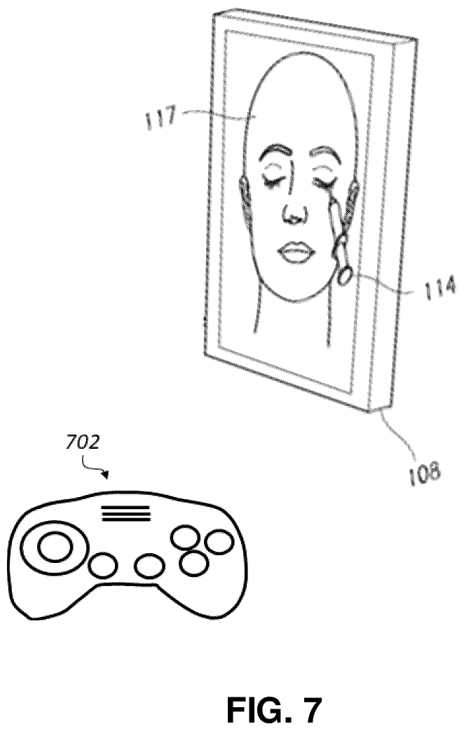

The training system can have an input device, such as a joystick or game controller for the Xbox, Nintendo Wii, or another video game and/or toy. FIG. 7 illustrates an example input device 702 that can be used with an injection training module disclosed herein on a display device 108, such as a PC tablet, a smartphone, or others. The input device 702 can be connected to the training module via a wired or wireless connection. The input device can be in electronic communication with the processor for the training modules.

The input device 702 can be used instead of a syringe and/or training apparatus to specify to the software the relationship between the computer images of a virtual syringe 114 and a virtual training apparatus or a patient 117. As shown in FIG. 7, a user can manipulate the input device 702 (for example, pushing one or more buttons, changing an angle or position of a pivoted stick, or otherwise) to control one or more features of the training modules. The user can manipulate the input device 702 to navigate the computer image of the training apparatus or patient 717, such as rotating, zooming, moving, and the like. The user can also optionally use the input device 702 to add and/or remove layers of anatomy in the virtual training apparatus or patient 717. The user can also manipulate the input device 702 to manipulate the computer image of the syringe 714, such as rotating, zooming, and/or moving the syringe relative to the training apparatus or patient 717, and/or performing a simulated injection with the computer image of the syringe 714. The input device 702 can have separate controls and/or hand gesture(s) for controlling the computer images of the training apparatus or patient 717 and the syringe 714, individually and/or simultaneously.

The input device such as the input device 702 shown in FIG. 7, can allow the anatomy and training modules to be used without the physical training apparatus and/or syringe. Allowing the training module to be used independent of the physical training apparatus (or real patient) and the syringe can be useful for providing simulated training injections and/or demonstrations, for example, in a trade show, a client pitch meeting, sales demonstration of the medication and/or the injection system or platforms, or others. The user of the training system may not need to carry the training apparatus and/or syringe when traveling to various locations to use the training system. The user can also learn injection procedures without needing access to the training apparatus and/or syringe. Learning injection procedures from the training modules with the input device or with just the training module can reduce a training cost compared to using the training system including the training apparatus and/or syringe, and/or allow the user to be trained while on the road. The input device and/or training module can be used for tradeshows where a user can compete with other users based on injection scoring. The input device and/or training module can also be used for presentations to reflect the anatomy and labels of the patients.

### INJECTION TRAINING SERVICE PROVIDER SYSTEM

### Matching Qualified Injection Providers With Physicians

Injection service provider systems and methods can keep records of injection training and/or certification of injectors (such as nurses) and facilitate collaboration between physicians and trained and/or certified injectors.

A trainee, such as a nurse, can receive injection training using embodiments of the injection training platforms described herein. The trainee can learn and/or improve injection skills in one or more injection techniques, and/or build a training and/or certification profile. A physician looking for a nurse familiar and/or experienced in certain injection techniques and/or products can view the profiles on the service provider system. The profile can provide information about the experience, training received, test scores, certification, and/or accuracy information of the trainee. The injection service provider can provide a database of qualified, trained, and/or certified injection providers for physicians searching for staff members. The trainee can use the database to promote himself or herself as a qualified injection provider.

### Matching Physicians and/or Injection Providers With Patients

The injection service provider can provide a database of qualified, trained, and/or certified injection providers, such as physicians and nurses, for patients who are searching for a physician or a nurse for certain injections. The injection service provider system is configured to allow patients to find and make reservations with the physicians and/or nurses listed in the system. The system can be configured to filter the list of physician and/or nurses based on the patient's location, price range, availability, and/or other filters.

The system can have features that are configured to encourage the patient to return to the same physician, nurse, and/or clinic. The physician and/or nurse can rate the patient after performing the injection procedure. The service provider system and/or the injection service provider can offer incentives, such as discounts, to patients who post a selfie and/or send the selfie to the injection service provide, and/or for booking the next appointment with the same injection service provider. The physician can review the selfie and/or examine the patient in person to check for complications. The injection syringes can record data of the injection procedures performed on the same patient. The physician and/or nurse can have access to the records of prior injection procedures upon the patient's return and apply substantially the same injection procedure to the same. The record of past injection procedures can promote repeatability of an injection procedure that is effective for the patient.

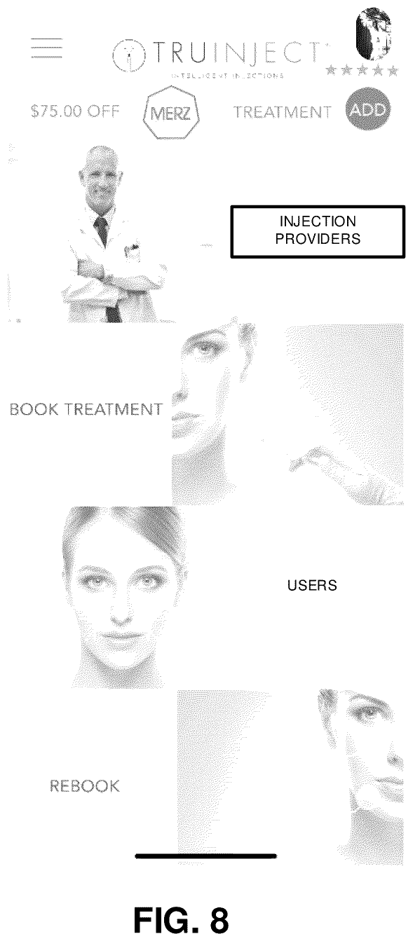

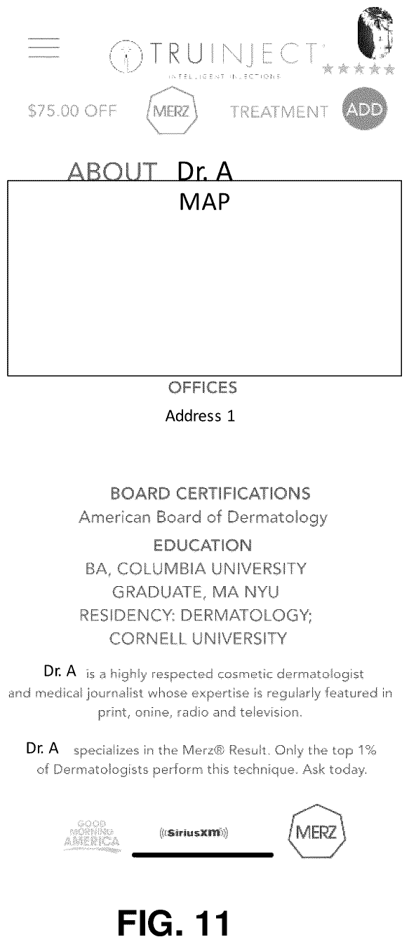

FIGS. 8-28 illustrate an example injection service provider application for matching physicians and/or injection providers with patients. As shown in FIG. 8, the application can include options for injection providers and patients, and for the patients to book treatment (“BOOK TREATMENT”) and to rebook for treatment (“REBOOK”).

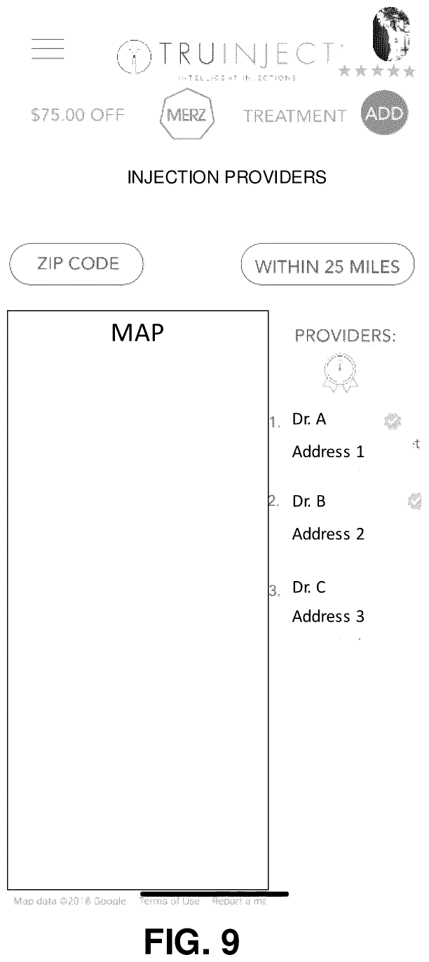

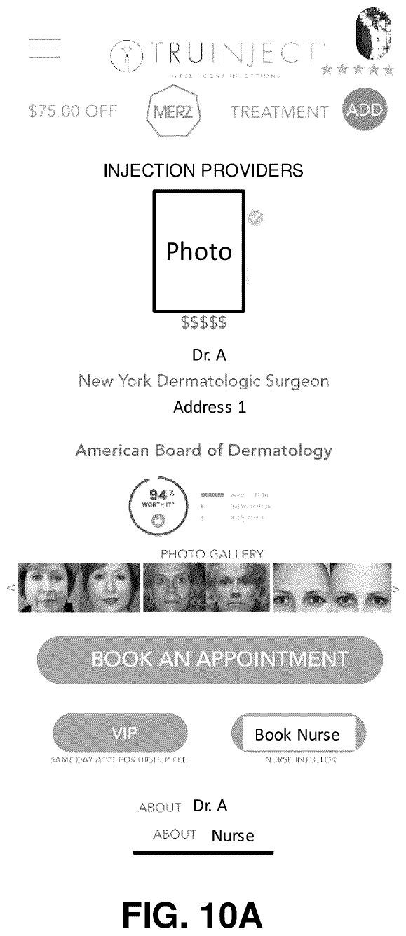

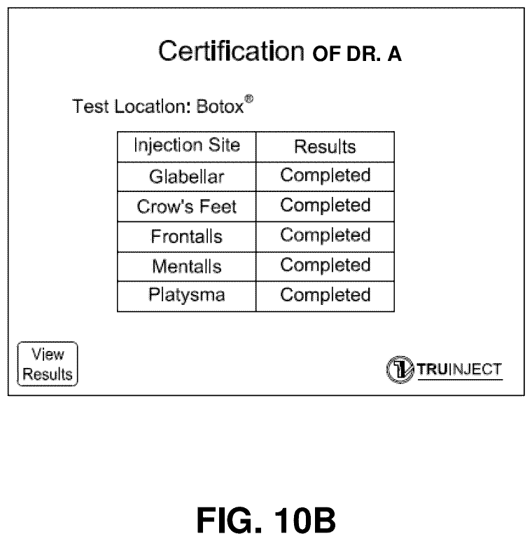

FIG. 9 illustrates the injection providers interface. The application can display nearby injection providers, such as based on the user's location. The application can also optionally display injection providers using other criteria, such as the injection providers'ratings, injection test scores, rankings, certification (see, for example, FIG. 10B), or others.

FIGS. 10A-B and 11 illustrate one of the injection providers, Dr. A. The application can provide various information about Dr. A, such as qualification, injection test scores, certification (FIG. 10B), services provided, address and/or maps, and/or snapshots of Dr. A's patients who have received injections from Dr. A. In some embodiments, patients or users can select injection providers based on the injection providers'accuracy of the training injections and/or the service provider's certifications for particular injections. As shown in FIG. 10A, the application can also provide a plurality of options for the user to use Dr. A's services, such as to book an appointment with Dr. A, use a VIP program, and/or book a procedure with a nurse (such as a registered nurse) at Dr. A's office.

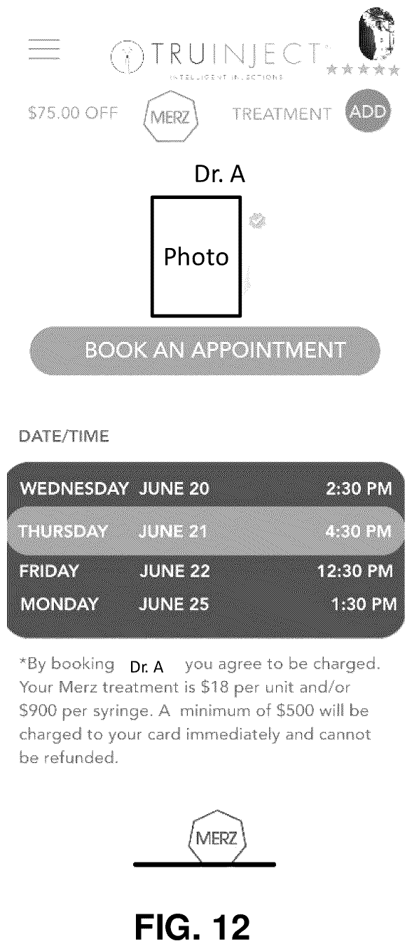

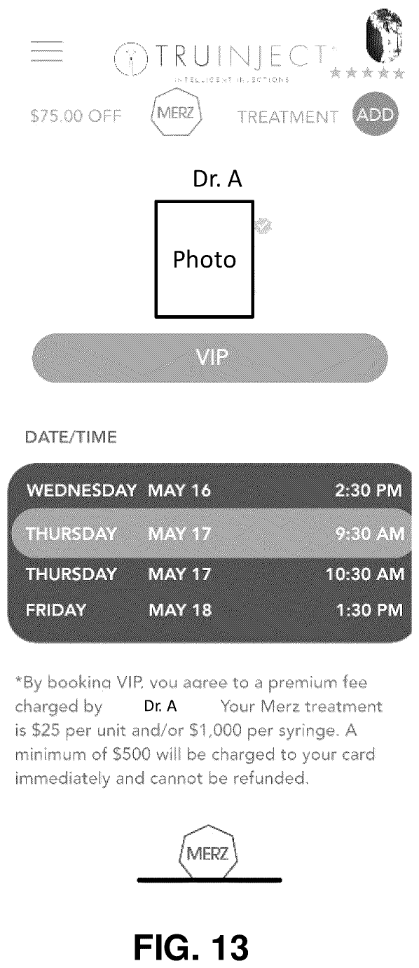

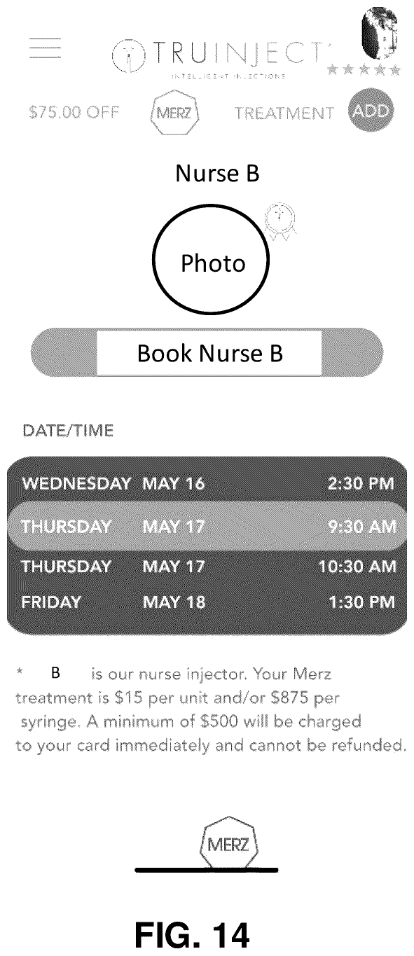

FIG. 12 illustrates a user interface when the user attempts to book an appointment with Dr. A. FIG. 13 illustrates a user interface when the user attempts to book a VIP appointment with Dr. A. The application can show Dr. A's availability. Using the VIP service can allow the user to access earlier appointment slots than the appointment slots for regular treatments (for example, for a higher fee for Dr. A's service). FIG. 14 illustrates a user interface when the user attempts to book an appointment with Nurse B who works at Dr. A's office.

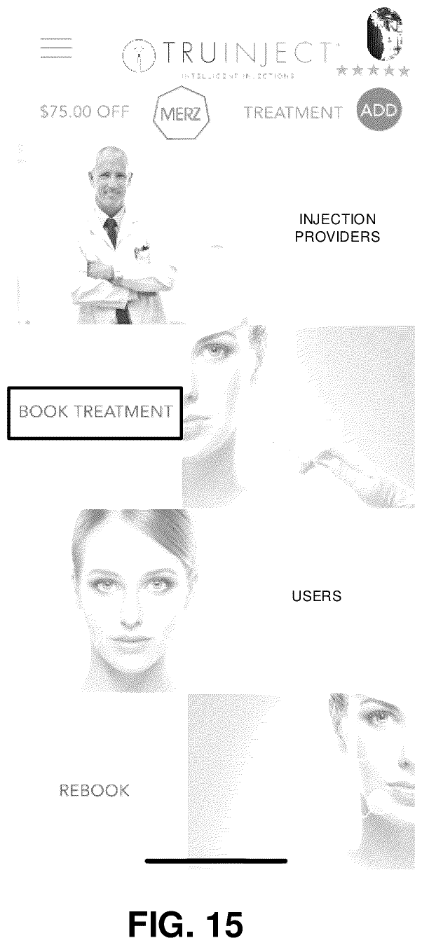

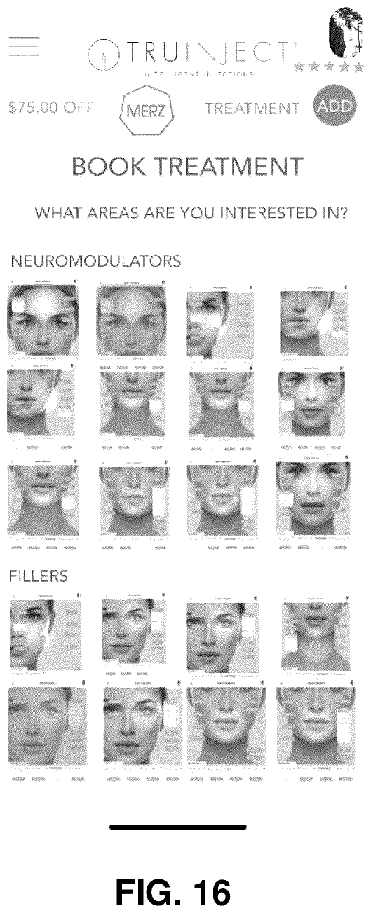

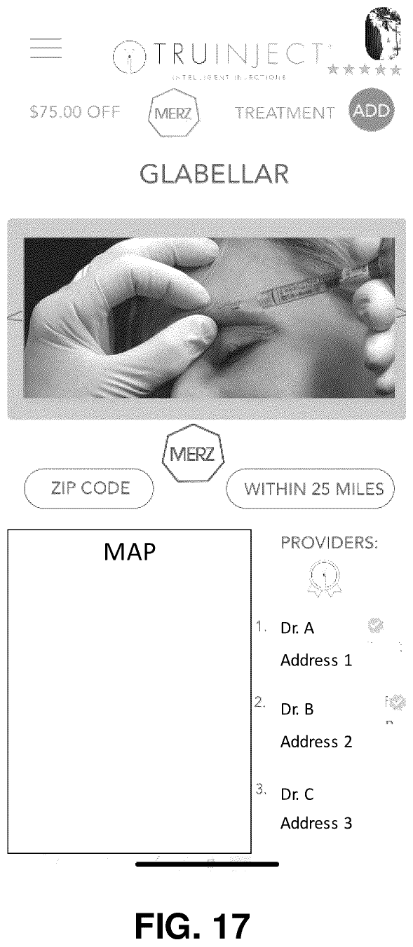

As illustrated in FIGS. 15-17, the user can choose to look for an injection provider based on the type of injection treatment rather than the proximity of the injection providers. The user can select “BOOK TREATMENT” in the main menu to be directed to a plurality of injection procedures, such as shown in FIG. 16. The user can select one of the treatments shown in the user interface, such as the glabellar procedure shown in FIG. 17 and review a video on any of the treatments. The application can provide a list of nearby injection providers who performed the procedure of interest. For example, the application can list the injection providers according to distance from the user and/or other criteria, such as ratings of the injection providers.

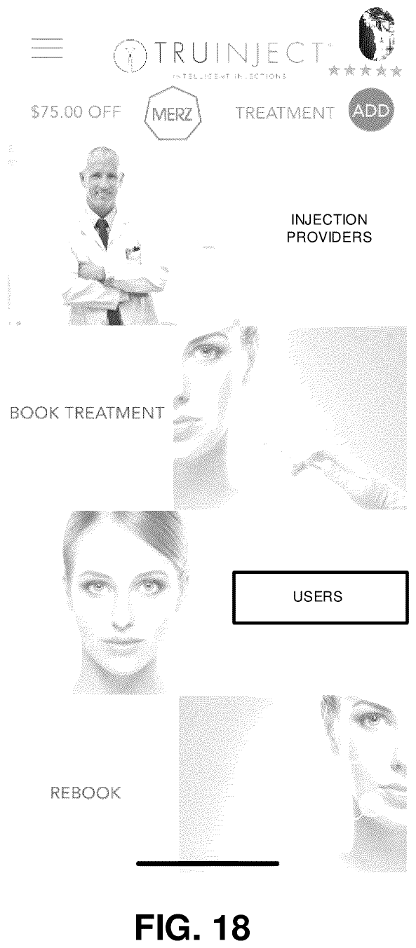

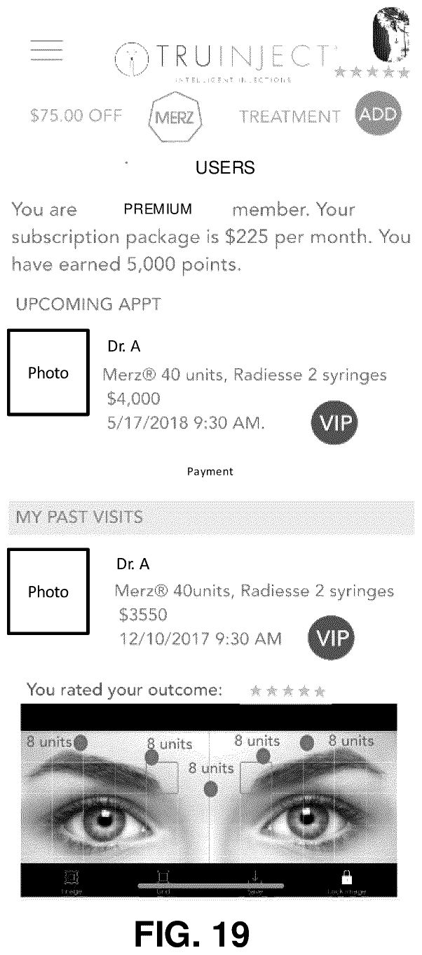

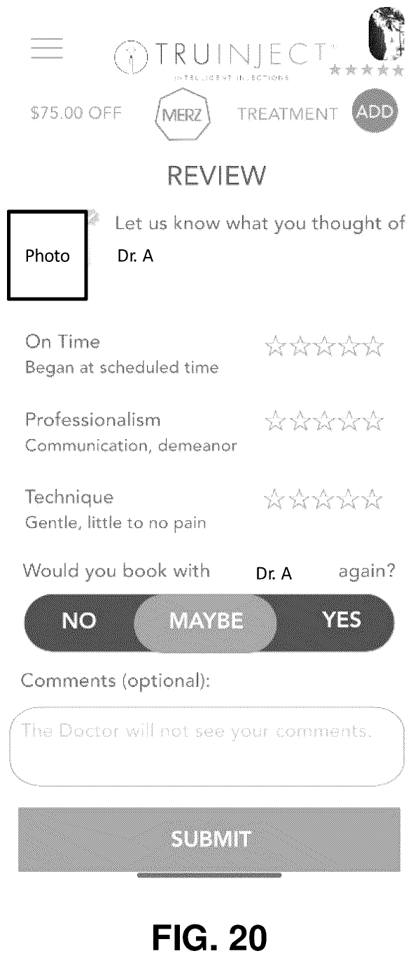

As shown in FIG. 18-20, the application can also allow access of the profiles of the users of the application, such as by selecting to view the users on the main menu. As shown in FIGS. 19 and 26, the user can view the user's own past and/or upcoming appointments on the application. The user can use the application to pay for the upcoming appointments. The user can also rate the outcome of the past treatments on the application. Injection information relating to the user's past treatments can be recorded using the electronics on the injection tool, such as described in U.S. Publication No. 2018/0211562. The injection information can include, for example, the location of the injection, the volume of products injected, or otherwise. In some embodiments, the user can also rate the technique and/or performance of the injection provider, such as shown in FIG. 20.

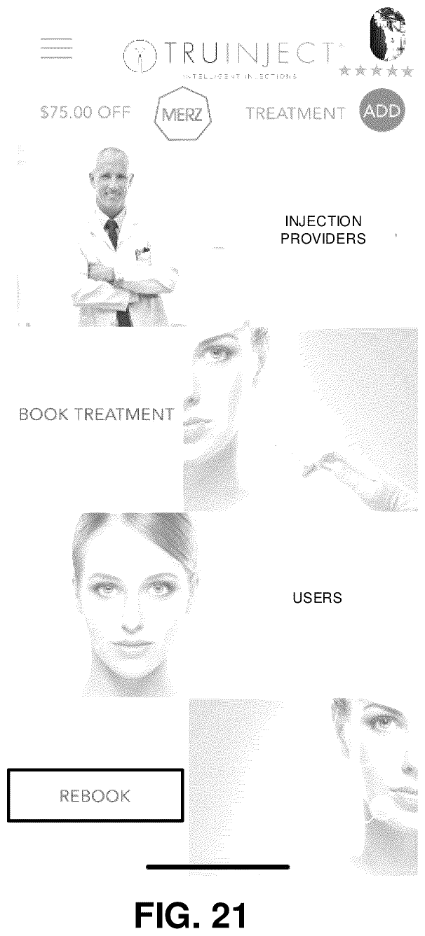

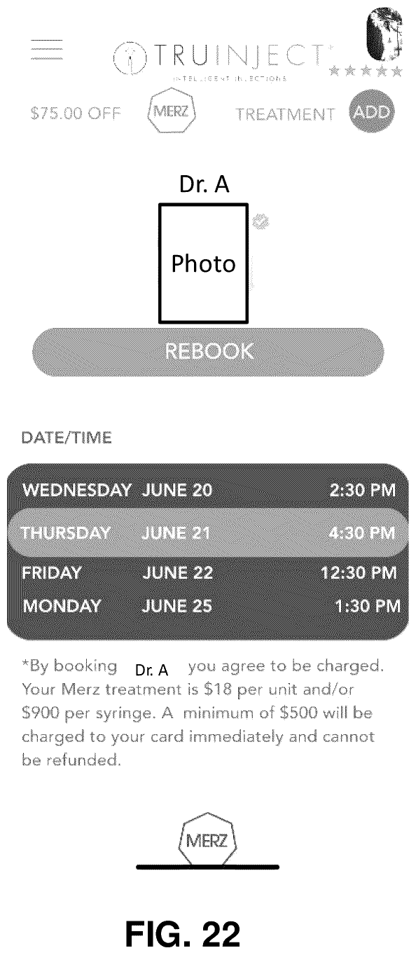

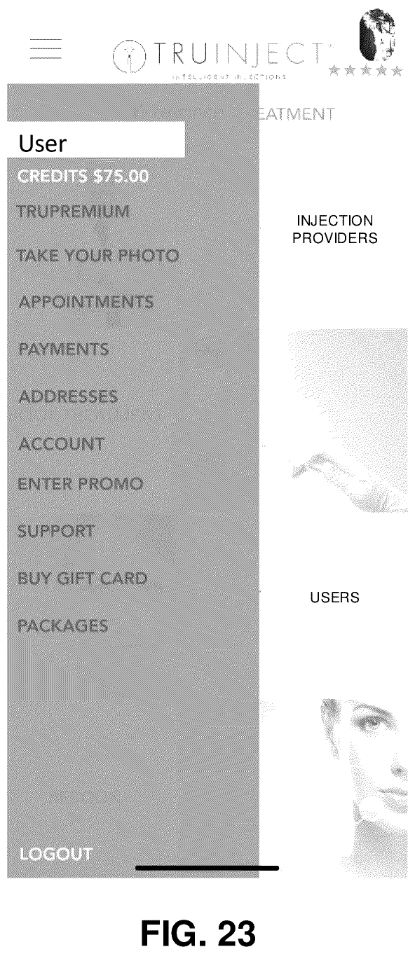

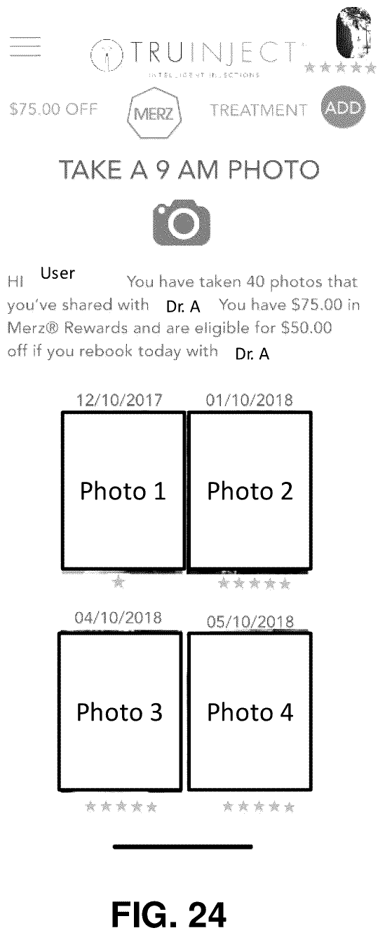

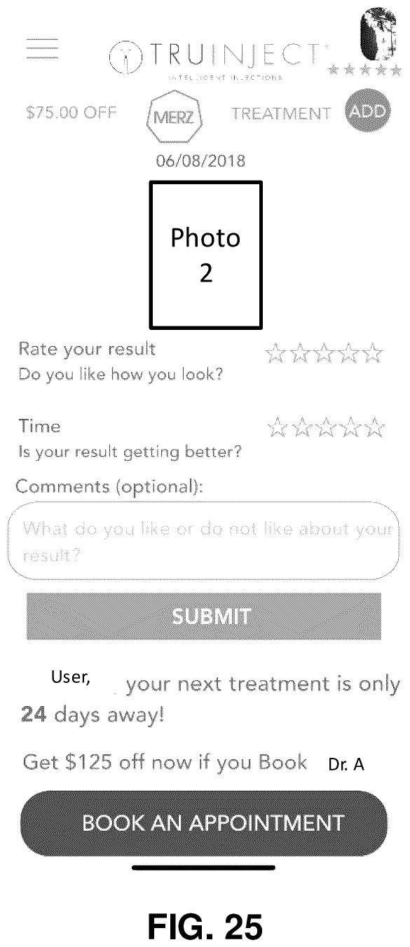

As shown in FIGS. 21-28, the application can allow a user to rebook a treatment with the same injection provider, such as by selecting “REBOOK” on the main menu. For example, the user can rebook a treatment with Dr. A, as shown in FIG. 22. The user can also optionally upload one or more photos for sharing with Dr. A to be eligible for discounts when rebooking with Dr. A, such as shown in FIGS. 23 and 24. The user can also rate how the user looks over time after the treatment, such as shown in FIG. 25. The user can get “rewarded” for posting pictures at certain time stamps, such as on the 7th day, the 30th day, and the 60th day. The photos can be rated by the user based on the satisfaction level of the user with the outcome. The photos (for example, with the injection provider's watermarks or otherwise) can be uploaded by injection providers, at the user's consent, or by the user, on a social media platform to promote the injection provider and/or the product. The photos can be stored with the user's account on the application to allow the user to review the photos and select the user's favorite injection outcome. If the patient was most satisfied with a particular treatment, the injection provider can use the same amount and location for subsequent treatment, such as by using the injection points from previous appointments (such as shown in FIG. 26) that are stored in the application.

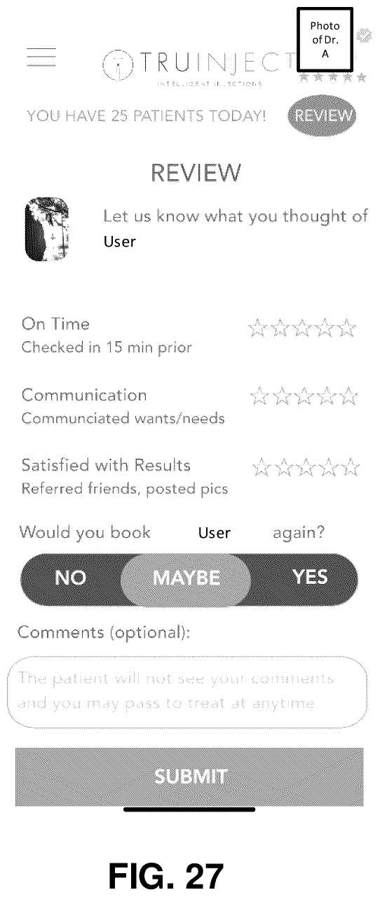

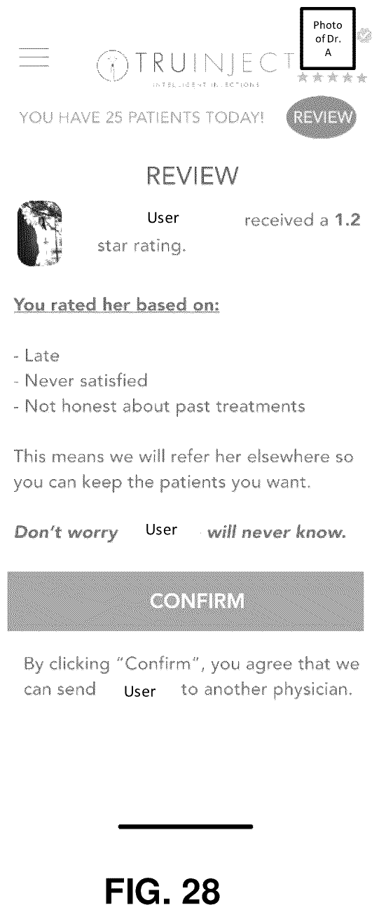

The application can also allow the injection provider to rate the patient or user, such as shown in FIGS. 27 and 28. In some embodiments, the application provider can allow the injection provider to rate and/or instruct the application to direct the user to another injection service provider anonymously. When the injection service provider gives a rating to a user that is below a predetermined thresholder, the application can direct the user to other injection service providers.

The application can also allow manufacturers of the treatment products to promote their products, and/or provide discounts to users who purchase those products via the application. In some embodiments, the manufacturers can offer the products in bundles or packages on the application.

### Combination and/or Subcombination Embodiments

Although features of the smart injection system or platform are described individually in various embodiments herein, a skilled artisan will appreciate that any one or more of those features described herein can be implemented on a smart injection system or platform.

It is to be understood that the various sensor and electronics, as well as the techniques and processes described with respect to each embodiment disclosed herein can be used with and integrated to other embodiments disclosed herein as would be readily understood by a person of skill in the art reading the present disclosure.

### Terminology

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Any methods disclosed herein need not be performed in the order recited. The methods disclosed herein include certain actions taken by a practitioner; however, they can also include any third-party instruction of those actions, either expressly or by implication. For example, actions such as “inserting the testing tool” include “instructing insertion of a testing tool.”

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. In some embodiments, the computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware (e.g., ASICs or FPGA devices), computer software that runs on general purpose computer hardware, or combinations of both. Various illustrative components, blocks, and steps have been described above generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a general purpose processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. An injection training platform, comprising:
a computing system having at least one processor and a memory device, the computing system configured to receive and process inputs relating to locations of an injection tool relative to a training anatomical model so as to output at least a virtual training anatomical model;
the computing system configured to apply one or more visual filters to the virtual training anatomical model so that the computing system further outputs to an augmented reality viewing system at least one of training technique instructions or an underlying anatomy overlaying the virtual anatomical model, wherein the one or more filters comprises ethnic-based anatomical variations, and
a display unit in electrical communication with the computing system, wherein the display unit displays a depiction of the training injection technique.
